(19) United States
(12) Patent Application Publication
(10) Pub. No.: US 2003/0060876 A1
(43) Pub. Date: Mar. 27, 2003

(54) Graft delivery system

(76) Inventors: Amir Loshakove, Moshav-Bazra (IL); Ido Kilemnik, Herzelia (IL); Ofer Nativ, Rishon-Lezion (IL)

Correspondence Address: William H Dippert, Reed Smith, 29th Floor, 599 Lexington Avenue, New York, NY, 10022-7650, US

(21) Appl. No.: 10/221,982

(22) PCT Filed: Jan. 24, 2001
(86) PCT No.: PCT/IL01/00069

(51) Int. Cl. A61F 2/06

(52) U.S. Cl. 623/1.23

(57) ABSTRACT

A graft delivery system (302), having a tubular element for delivering a graft (510) through a bore (320) thereof and having a delivery end (410) and the end being prone to distortion and at least one collar (300) removably encircling the delivery end, which collar prevents the distortion.

### RELATED APPLICATIONS

This application claims the benefit under 119(e) of U.S. provisional application 60/254,689. This application is a continuation in part of PCT applications PCT/IL99/00284, PCT/IL99/00670, PCT/IB00/00310, and PCT/IL00/00609. The disclosures of all of these applications, which are filed by applicant Bypass and designate the US, are incorporated herein by reference.

### FIELD OF THE INVENTION

The present invention relates to devices for delivering a graft for an anastomosis.

### BACKGROUND OF THE INVENTION

Anastomotic connections may be made using sutures or using a dedicated anastomotic connector. In a typical connector application, the connector is mounted on an end of a tube and a graft is brought through the tube to the end of the tube on which the connector is mounted. Various, inconvenient methods for bringing the graft through the tube, such as pushing the graft through the tube, are commonly performed.

Some types of anastomotic connectors are super-elastic. An anastomotic delivery system with a connector pre-loaded may be stored for a considerable period of time, before it is actually used.

### SUMMARY OF THE INVENTION

An aspect of some embodiments of the invention relates to loading a graft into a tubular graft delivery system. In an exemplary embodiment of the invention, the graft is pulled through the tubular delivery system by a retractable prong. Optionally, the graft is provided thorough an aperture in the side of the graft delivery system and is pulled through the tube so that one end of the graft extends out of one end of the graft delivery system. Optionally, the retractable prong is mounted on a shaft having an outer diameter slightly smaller than the inner diameter of the path for the graft in the graft delivery system.

In an exemplary embodiment of the invention, the shaft is bent, so that when the puller is inserted through the end of the graft delivery system, the prong end of the graft exits form the aperture in the side of the delivery system. Alternatively or additionally, the shaft is formed of a flexible material, which allows it to be straightened by the inner bore of the delivery system when it is pushed through the delivery system.

An aspect of some embodiments of the invention relates to protecting a graft from being damaged as it is loaded into a delivery tube. In an exemplary embodiment of the invention a removable graft guide is mounted on the delivery tube to prevent contact between the graft and harmful parts (e.g., corners, edges) of the delivery system.

An aspect of some embodiments of the invention relates to preventing distortion of an anastomosis connector delivery system. In an exemplary embodiment of the invention, a connector is pre-loaded into the delivery system. If the connector is made of an elastic, super-elastic or shape memory material, is pre-stressed so that it applies radial force against the tube: Optionally, distortion-causing forces applied to a tube of the delivery system by the connector are counteracted by at least one collar mounted on the tube. Optionally, the at least one collar is mounted at or about the axial position of the connector. Alternatively or additionally, the distortion-causing forces are caused by the design of the system, which includes pre-weakened portions for easy axial splitting of the system. Such pre-weakened portions may pre-dispose the delivery system for distortion during storage even if no connector is present.

In an exemplary embodiment of the invention, the collar comprises a split collar for side removal from the connector delivery system. Optionally the split collar is connected to a graft guide so that both can be removed from the delivery system in a single step. Optionally, the split collar is rough on its outside, to engage a graft when such a graft is everted over the split collar. Alternatively or additionally, the split collar extends forward and/or radially to protect the graft from sharp tips of an anastomotic connector, if the tips extend out of the connector delivery system.

In an exemplary embodiment of the invention, the collar comprises a ring that encircles the delivery system. Optionally the collar can be attached to the shaft of the retractable prong, for example forming a handle for the shaft.

Optionally, there are two collars that encircle the delivery system. One is for long term distortion prevention, for example for use during storage, and is removed sometime near the loading of the graft on the delivery system. The other collar remains mounted on the graft delivery system during at least part of the process of loading the graft. In the embodiment where the collar is attached to the shaft of the retractable prong, using the retracting prong removes the collar from encircling the delivery system.

There is thus provide din accordance with an exemplary embodiment of the invention, a medical graft delivery system, comprising:

a tubular element for delivering a graft through a bore thereof and having a delivery end, said end being prone to distortion; and

at least one collar removably encircling said delivery end, which collar prevents said distortion. Optionally, said tube defines an aperture in its side, thorough which a graft may be inserted. Alternatively or additionally, said tube comprises weakened portions at or adjacent said delivery end. Alternatively or additionally, said system comprises an anastomotic connector preloaded in said delivery end and applying outward forces against said end.

In an exemplary embodiment of the invention, said at least one collar comprises at least two collars. Alternatively or additionally, said at least one collar comprises a split collar adapted to be removed from a side of said delivery system. Optionally, said split collar has a roughened outer surface, adapted to engage a graft.

In an exemplary embodiment of the invention, said at least one collar comprises a complete collar adapted to be removed axially from said delivery end. Optionally, said at least one collar is integrated with a handle of a vessel puller adapted to pull a vessel through said graft delivery system.

In an exemplary embodiment of the invention, said at least one collar is integrated with a graft guide, said guide adapted to prevent damaging contact between said graft and said delivery system, during a loading of said graft into said delivery system.

In an exemplary embodiment of the invention, the delivery system is provided in a sterile package.

There is also provided in accordance with an exemplary embodiment of the invention, a removable graft guide for a graft delivery system, comprising:

a body removably mounted on said delivery system; and

a guide portion adapted to fit in an aperture in said graft delivery system and prevent contact between a graft inserted through said aperture and damaging parts of said delivery system. Optionally, said body is adapted to be mounted on an outside of said delivery system. Alternatively or additionally, said body comprises a collar for preventing distortion of a delivery end of said delivery system.

There is also provide din accordance with an exemplary embodiment of the invention, a vessel puller, comprising:

a shaft adapted to be inserted in a bore of a tubular graft delivery system having a delivery end;

a vessel engager mounted on one end of the shaft and adapted to engage a tip of a graft; and

a handle attached to another end of said shaft. Optionally, said handle is adapted to enclose said delivery end. Alternatively or additionally, the shaft is longer than a distance between an aperture in the side of said bore and said delivery end, such that said handle can be comfortably held by a person while a graft is inserted, by said person, through said aperture to be engaged by said vessel engager.

In an exemplary embodiment of the invention, said vessel engager is adapted to engage a graft end from a side of said engager. Optionally, said shaft is flexible. Alternatively or additionally, said shaft is pre-bent. Alternatively or additionally, said puller comprises a shaft bending control for selectively bending said shaft.

### BRIEF DESCRIPTION OF THE FIGURES

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements or parts which appear in more than one figure are preferably labeled with a same or similar number in all the figures in which they appear, in which:

FIG. 1 shows a design of a graft puller with cap, in accordance with an exemplary embodiment of the invention;

FIG. 2A shows the tip of the graft puller of FIG. 1 in a retracted position;

FIG. 2B shows the tip of the graft puller of FIG. 1 in an extended position;

FIG. 3 shows the graft puller loaded inside a graft delivery system, in accordance with an exemplary embodiment of the invention;

FIG. 4 shows a collar/graft guide in association with a graft delivery system, in accordance with an exemplary embodiment of the invention; and

FIG. 5 is a cut-through view of the collar/graft guide mounted on the graft delivery system, in accordance with an exemplary embodiment of the invention.

### DETAILED DESCRIPTION OF EXEMPLARY EMBODIMENTS

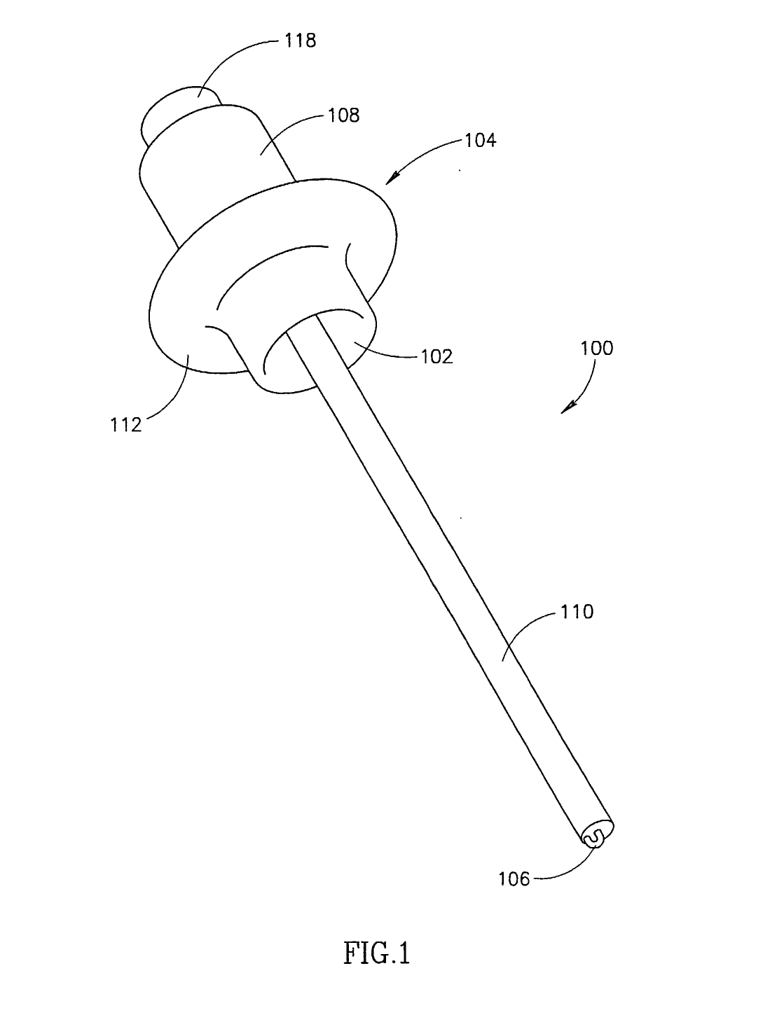

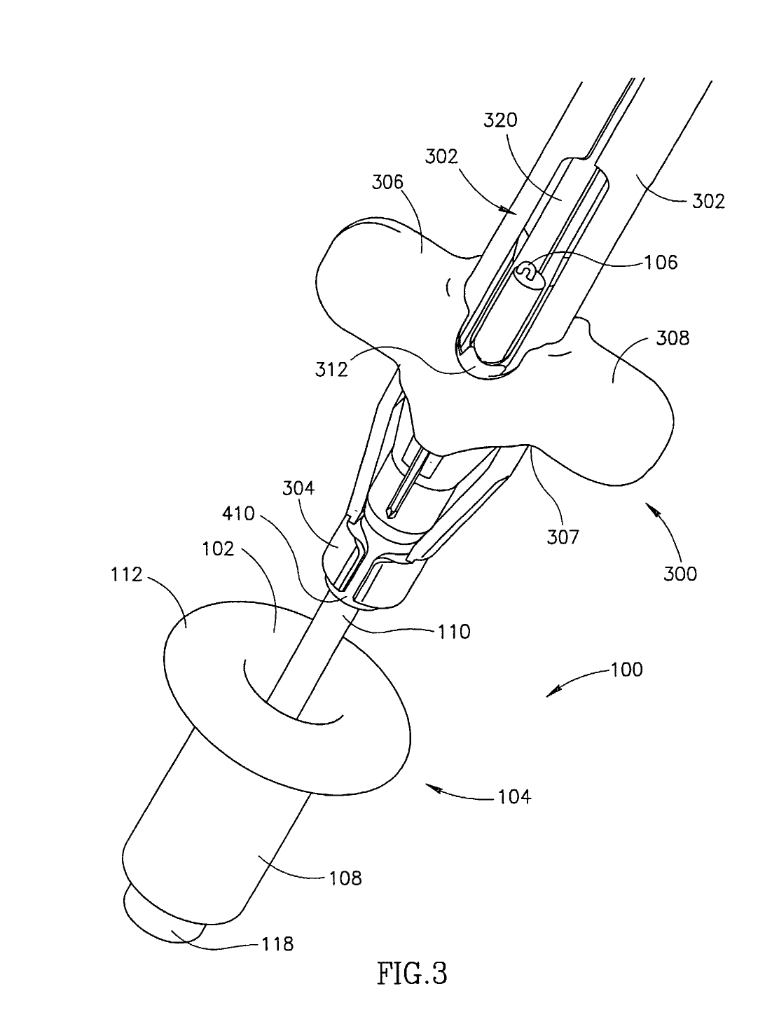

FIG. 1 shows an exemplary design of a graft puller 100, for use in a tubular graft delivery system (described in FIG. 3), for pulling a graft through an inner bore of the graft delivery system. In an exemplary embodiment of the invention, graft puller 100 includes an elongate shaft 110 having a graft engaging portion at one end thereof, for example, a retractable prong 106. Optionally, a handle 104 is provided on the other end of shaft 110. Optionally, handle 104 includes an inner cup 102 adapted to engage the tip of a graft delivery system (described below), for example for keeping the delivery system from being distorted or to keep the graft puller from moving within the delivery system. Optionally, a flange 112 and/or a handle body 108 are provided on handle 104, to assist in grasping graft puller 100.

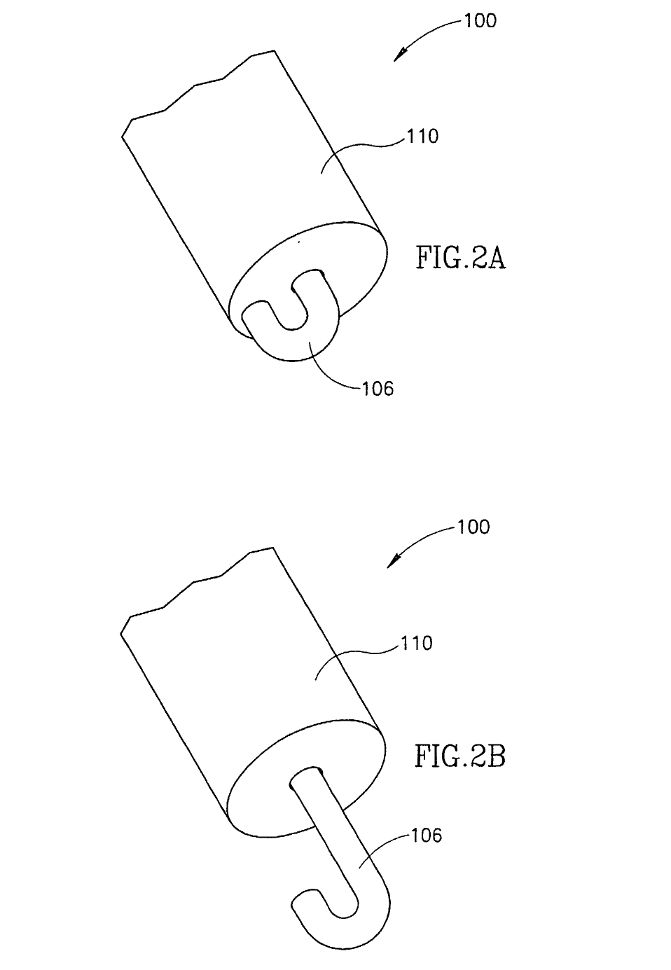

FIGS. 2A and 2B show retractable prong 106 in a retracted and an extended position, respectively. In FIG. 2A, prong 106 is retracted towards shaft 110, potentially engaging a graft tip between the prong and the shaft. In FIG. 2B, prong 106 is extended, to allow a graft tip to be placed between the prong and the shaft.

In an exemplary embodiment of the invention, the prong is a spring loaded-prong having a resting state in the retracted position. For example, a button 118 (FIG. 1) may be depressed in order to momentarily extend prong 106, as in FIG. 2B. Alternatively, other operation methods may be used, for example, twisting of button 118 (or another control) to axially translate the prong.

Optionally, a space is defined between the tip of prong 106 in its most retracted position and shaft 110, to prevent damaging of the graft (e.g., by pinching). Alternatively or additionally, the face of shaft 110 may be shaped to better engage the graft, when the prong is retracted. Alternatively or additionally, especially in a spring loaded prong, the degree of force that can be applied by the prong is limited by the spring, to prevent damaging of the graft. Optionally, prong 106 is made elastic, to limit the applied force.

Alternatively to using the retractable prong design shown in FIGS. 1, 2A and 2B, other vessel engaging means may be provided, for example, a forked clamp that splits upon pressing button 118, and clamps upon a graft when button 118 is released. An alternative design is a loop into which the graft tip is inserted. Optionally, the loop retracts into an axial opening in shaft 110, to immobilize the graft. The face of the opening is optionally curved, to prevent damaging of the graft.

FIG. 3 shows graft puller 100 and handle 104 loaded into a delivery system 302. A collar/graft guide 300, which is explained below, is also shown, mounted on delivery system 302.

Delivery system 302, in one exemplary embodiment of the invention, comprises a hollow tube with controls (not shown) at one end and an opening, through which shaft 110 is inserted, at a delivery end 410. Delivery system 302 includes an aperture 320 in its side, through which a graft can be inserted.

In an exemplary embodiment of the invention, shaft 110 can be bent or is pre-bent, so that when inserted through the inner bore of system 302, prong 106 exits through aperture 320. Optionally, shaft 110 is made flexible, for example of a suitable plastic. Alternatively or additionally, shaft 110 can be bent, for example, using a pull-string or a push rod in the shaft, as known in the art of bending catheters, for example. In an exemplary embodiment of the invention, shaft 110 is between 5 and 21 cm long, possibly between 7 and 13 cm long. Shaft 110 may have an outer diameter substantially the same as or smaller than the inner diameter of the bore in system 302.

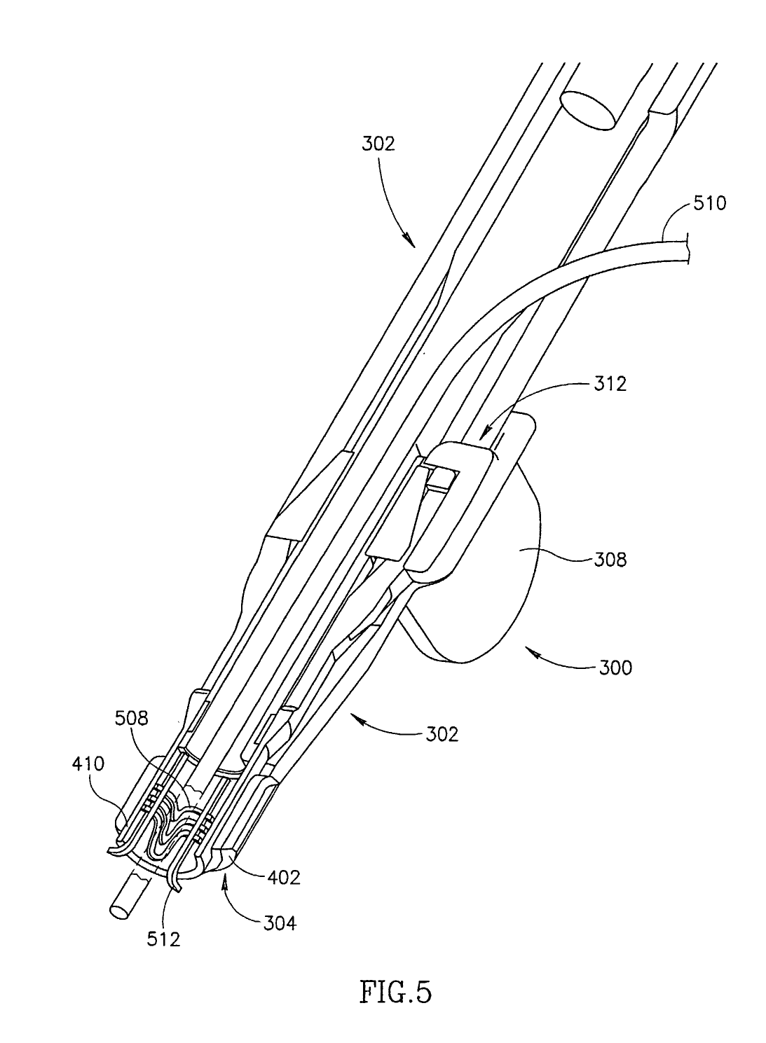

In an exemplary embodiment of the invention, delivery system 302 is provided preloaded with an anastomotic connector (see FIG. 5) inside the system and with cup 102 over end 410. After system 302 is removed from its packaging, handle 104 is retracted, freeing cup 102 from delivery system 302 and bringing prong 106 into alignment with aperture 320. A graft is inserted through aperture 320 and engaged by prong 106. Handle 104 is then further retracted, pulling shaft 110 with the graft grasped by it, out through the tip of delivery system 302. The graft may then be everted and/or mounted on the anastomosis connector, as described, for example, in the above related applications. The loaded delivery system is then inserted into a delivery system handle (also shown in the related applications) that includes a homeostatic valve at its end near a target blood vessel. Previously, a punch was brought through the valve to punch a hole in the blood vessel. This punch is replaced by the loaded delivery system. After the anastomosis connection is performed, the delivery system is removed, for example, by splitting it off the graft.

Cup 102, which engages end 410, may have a restraining function of preventing end 410 of delivery system 302 from being distorted during storage. In the exemplary embodiment shown, the restraining element (e.g., cup 102 or an alternative implementation) is provided integral with handle 104 of graft puller 100, allowing graft puller 100 to be pre-loaded into delivery system 302, during storage. Alternatively, a separate restraining element may be provided, for example, a removable collar. The inner cross-section of cup 102 (or of another collar) may be circular or it may be other, for example, hexagonal. Alternatively to an outer collar, in an exemplary embodiment of the invention, a thin, possibly metal, collar is provided inside delivery system 302, between the connector (508, FIG. 5) and the outer tube of system 302.

Optionally, an additional (or alternative) collar 300 is provided for delivery system 302. In an exemplary embodiment of the invention, additional collar 300 is in a form that allows the collar to be removed from the side of delivery system 302, by moving it radially, rather than by moving it in an axial direction. Optionally, collar 300 remains in place during at least part of the loading of the graft, thus providing a temporary distortion prevention function.

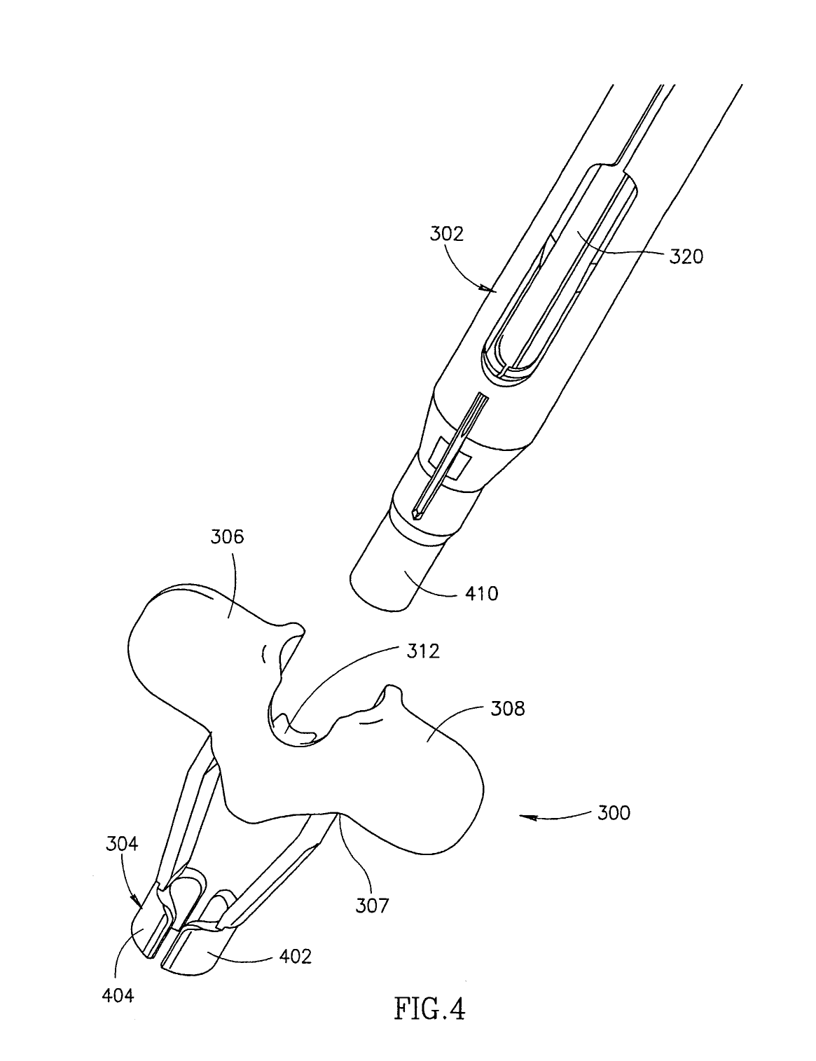

FIG. 4 shows collar 300 separate from delivery system 302, in accordance with an embodiment of the present invention. FIG. 4 also shows a clear view of end 410 of delivery system 302, which overlies an anastomosis connector (508, FIG. 5). Collar 300 includes a two part split collar 304 having separate runnels 402 and 404. In other embodiments, the runnels may be connected on one side, at the collar. In an exemplary embodiment of the invention, runnels 402 and 404 include elongate arms that distance the end-engaging portion from a collar body 307. Collar body 307 may include two wide tabs 306 and 308, which tabs may be used for applying force to remove the collar and/or for ease in holding delivery system 302. In an exemplary embodiment of the invention, collar body 307 is molded to engage graft delivery system 302. Optionally, collar 300 includes a graft guide 312.

FIG. 5 is a cut-through view of delivery system 302 with collar 300 mounted thereon, showing the protective function of guide 312, with respect to a graft 510.

In an exemplary embodiment of the invention, graft guide 312 protects the graft from contacting sharp edges or other potentially damaging parts of delivery system 302, especially when the graft is being pulled through delivery system 302. Optionally, removable graft guide 312 is provided without relation to the collar function of collar 300, for example, absent collar portion 304.

Also shown in FIG. 5 is an anastomotic connector 508 having forward spikes 512, which may apply radial forces that can deform tip 410 of graft delivery system 302. Runnels 402 and 404 (shown in FIG. 4) apply pressure to tip 410 to prevent such deformation.

In an exemplary embodiment of the invention, graft 510 is everted over collar portion 304. Optionally, collar portion 304 is provided with a rough surface, to engage the graft, so that when spikes 512 of connector 508 are allowed to extend out and penetrate the graft, the graft does not move too much.

Alternatively or additionally, collar portion 304 protects the everted graft from spikes 512. The protection may be achieved, for example, by the tips of the spikes being covered by collar portion 304, in which case collar portion 304 may include slits for the spikes to pass through. Alternatively collar 304 extends radially more than the spike tips, thus preventing them for penetrating the graft.

Alternatively, collar 300 may be removed prior to eversion, for example, to allow the eversion to be performed on a smaller diameter tube (than of delivery system 302).

In an exemplary embodiment of the invention, the graft delivery system is made mostly or wholly of plastic. Optionally, the graft delivery system is disposable and is provided, as a kit, in a sterile packaging. Optionally, the kit includes usage instructions, for example on a separate piece of paper.

It will be appreciated that the above described methods and devices of vascular manipulation may be varied in many ways, including, changing the order of steps, which steps are performed inside the body and which outside, the order of making the anastomosis connections, the order of steps inside each anastomosis and the exact materials used for the anastomotic connectors. Further, in the mechanical embodiments, the location of various elements may be switched, without exceeding the sprit of the disclosure, for example, switching the moving elements for non-moving elements where relative motion is required. In addition, a multiplicity of various features, both of methods and of devices have been described. It should be appreciated that different features may be combined in different ways. In particular, not all the features shown above in a particular embodiment are necessary in every similar exemplary embodiment of the invention. Further, combinations of the above features, from different described embodiments are also considered to be within the scope of some exemplary embodiments of the invention. In addition, some of the features of the invention described herein may be adapted for use with prior art devices, in accordance with other exemplary embodiments of the invention. The particular geometric forms used to illustrate the invention should not be considered limiting the invention in its broadest aspect to only those forms, for example, where a circular lumen is shown, in other embodiments an oval lumen may be used.

Also within the scope of the invention are surgical kits which include sets of medical devices suitable for making a single or a small number of anastomosis connections. Measurements are provided to serve only as exemplary measurements for particular cases, the exact measurements applied will vary depending on the application. When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A medical graft delivery system, comprising:
a tubular element for delivering a graft through a bore thereof and having a delivery end, said end being prone to distortion; and
at least one collar removably encircling said delivery end, which collar prevents said distortion.

2. A system according to claim 1, wherein said tube defines an aperture in its side, thorough which a graft may be inserted.

3. A system according to claim 1, wherein said tube comprises weakened portions at or adjacent said delivery end.

4. A system according to claim 1, comprising an anastomotic connector preloaded in said delivery end and applying outward forces against said end.

5. A system according to any of claims 1-4, wherein said at least one collar comprises at least two collars.

6. A system according to any of claims 1-4, wherein said at least one collar comprises a split collar adapted to be removed from a side of said delivery system.

7. A system according to claim 6, wherein said split collar has a roughened outer surface, adapted to engage a graft.

8. A system according to any of claims 1-4, wherein said at least one collar comprises a complete collar adapted to be removed axially from said delivery end.

9. A system according to claim 8, wherein said at least one collar is integrated with a handle of a vessel puller adapted to pull a vessel through said graft delivery system.

10. A system according to claim 6, wherein said at least one collar is integrated with a graft guide, said guide adapted to prevent damaging contact between said graft and said delivery system, during a loading of said graft into said delivery system.

11. A system according to any of claims 1-4, wherein the delivery system is provided in a sterile package.

12. A removable graft guide for a graft delivery system, comprising:
a body removably mounted on said delivery system; and
a guide portion adapted to fit in an aperture in said graft delivery system and prevent contact between a graft inserted through said aperture and damaging parts of said delivery system.

13. A guide according to claim 12, wherein said body is adapted to be mounted on an outside of said delivery system.

14. A guide according to claim 12 or claim 13, wherein said body comprises a collar for preventing distortion of a delivery end of said delivery system.

15. A vessel puller, comprising:
a shaft adapted to be inserted in a bore of a tubular graft delivery system having a delivery end;
a vessel engager mounted on one end of the shaft and adapted to engage a tip of a graft; and
a handle attached to another end of said shaft.

16. A puller according to claim 15, wherein said handle is adapted to enclose said delivery end.

17. A puller according to claim 15, wherein the shaft is longer than a distance between an aperture in the side of said bore and said delivery end, such that said handle can be comfortably held by a person while a graft is inserted through said aperture, by said person, to be engaged by said vessel engager.

18. A puller according to claim 15, wherein said vessel engager is adapted to engage a graft end from a side of said engager.

19. A puller according to any of claims 15-18, wherein said shaft is flexible.

20. A puller according to any of claims 15-18, wherein said shaft is pre-bent.

21. A puller according to any of claims 15-18, comprising a shaft bending control for selectively bending said shaft.
